(19) United States
(12) Patent Application Publication
(10) Pub. No.: US 2009/0324635 A1
(43) Pub. Date: Dec. 31, 2009

(54) Caspofungin free of caspofungin impurity A

(76) Inventors: Ferenc Korodi, Debrecen (HU); Piroska Kovacs, Debrecen (HU); Chaim Eidelman, Tal-El (IL); Avi Tovi, Zurit (IL); Hagi Alon, Kibbutz Yehiam (IL)

Correspondence Address: KENYON & KENYON LLP, ONE BROADWAY, NEW YORK, NY, 10004, US

(21) Appl. No.: 12/459,198

(22) Filed: Jun. 25, 2009

Related U.S. Application Data
(60) Provisional application No. 61/133,184, filed on Jun. 25, 2008.
(60) Provisional application No. 61/133,602, filed on Jun. 30, 2008.
(60) Provisional application No. 61/174,289, filed on Apr. 30, 2009.
(60) Provisional application No. 61/188,385, filed on Aug. 7, 2008.
(60) Provisional application No. 61/139,873, filed on Dec. 22, 2008.

(51) Int. Cl. A61K 38/12; C07K 7/64; G01N 33/15; G01N 31/22; A61P 31/10

(52) U.S. Cl. 424/195.15; 530/321; 436/86; 436/15; 514/11

(57) ABSTRACT

Provided is caspofungin free of caspofungin impurity A, methods for preparation thereof and isolation of caspofungin impurity A.

### CROSS REFERENCE

The present invention claims the benefit of the following U.S. Provisional Patent Application Nos.: 61/133,184 filed Jun. 25, 2008; 61/133,602 filed Jun. 30, 2008; 61/174,289 filed Apr. 30, 2009; 61/188,385 filed Aug. 7, 2008; and 61/139,873 filed Dec. 22, 2008. The contents of these applications are incorporated herein by reference.

### FIELD OF INVENTION

The present invention relates to caspofungin free of caspofungin impurity A, methods for preparation thereof and isolation of caspofungin impurity A.

### BACKGROUND OF THE INVENTION

Caspofungin, 1-[(4R,5S)-5-[(2-Aminoethyl)amino]-N2-(10,12-dimethyl-1-oxotetradecyl)-4-hydroxy-L-ornithine]-5-[(3R)-3-hydroxy-L-ornithine]-pneumocandin B₀, of the following formula

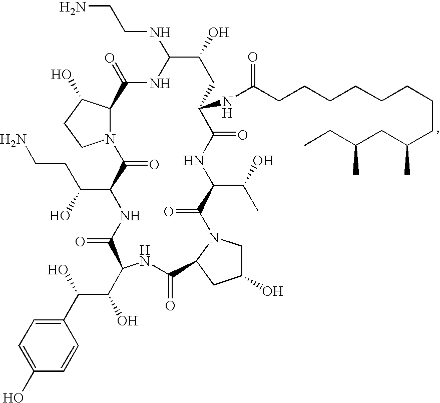

is a macrocyclic lipopeptide from the echinocandin family, a new class of antifungal agents that inhibits the synthesis of beta (1,3)-D-glucan, an integral component of the fungal cell wall. The echinocandin family is known to be useful in treating systemic fungal infections, especially those caused by Candida, Aspergillus, Histoplasma, Coccidioides and Blastomyces. They have also been found useful for the treatment and prevention of infections caused by Pneumocystis carinii which are often found in immunocompromised patients such as those with AIDS. Caspofungin shows additive or synergic antifungal activity with amphotericin B and triazoles.

Caspofungin is administrated as a diacetate salt and sold under the trade name Cancidas® by Merck & Co., Inc.

Caspofungin is a semi-synthetic product that can be prepared from Pneumocandin B₀ of the following formula

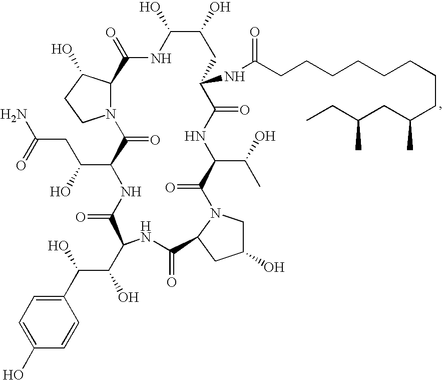

which is a natural product, obtained from sources such as fermentation reactions. The preparation of Pneumocandin B₀ is disclosed in several publications such as U.S. Pat. No. 5,194,377 and U.S. Pat. No. 5,202,309.

Caspofungin and its pharmaceutical acceptable salts are known under the INN (International Nonproprietary Names) to be useful in treating fungal infections (see Merck Index, 13th edition, monograph no. 1899).

U.S. Pat. No. 5,378,804 describes Caspofungin and U.S. Pat. Nos. 5,552,521 and 5,936,062 describes synthetic approaches to prepare Caspofungin.

Like any synthetic compound, Caspofungin can contain extraneous compounds or impurities.

The Journal of Industrial Microbiology and Biotechnology 26, 216-211 (2001) describes a transformation of Pneumocandin B₀ contaminated with its serine analogue to caspofungin containing serine analogue of caspofungin.

The serine analogue of Pneumocandin B₀ was also described in WO 2000/08197. However, isolation or characterization of the caspofungin serine analogue itself has not been provided in neither of the above mentioned publications.

Impurities in Caspofungin, or any active pharmaceutical ingredient (“API”), are undesirable and, in extreme cases, might even be harmful to a patient being treated with a dosage form containing the API.

The purity of an API produced in a manufacturing process is critical for commercialization. The U.S. Food and Drug Administration (“FDA”) requires that process impurities be maintained below set limits. For example, in its ICH Q7A guidance for API manufacturers, the FDA specifies the quality of raw materials that may be used, as well as acceptable process conditions, such as temperature, pressure, time, and stoichiometric ratios, including purification steps, such as crystallization, distillation, and liquid-liquid extraction. See ICH Good Manufacturing Practice Guide for Active Pharmaceutical Ingredients, Q7A, Current Step 4 Version (Nov. 10, 2000).

The product of a chemical reaction is rarely a single compound with sufficient purity to comply with pharmaceutical standards. Side products and by-products of the reaction and adjunct reagents used in the reaction will, in most cases, also be present in the product. At certain stages during processing of an API, it must be analyzed for purity, typically, by high performance liquid chromatography (“HPLC”) or thin-layer chromatography (“TLC”), to determine if it is suitable for continued processing and, ultimately, for use in a pharmaceutical product. The FDA requires that an API is as free of impurities as possible, so that it is as safe as possible for clinical use. For example, the FDA recommends that the amounts of some impurities be limited to less than 0.1 percent. See ICH Good Manufacturing Practice Guide for Active Pharmaceutical Ingredients, Q7A, Current Step 4 Version (Nov. 10, 2000).

Generally, side products, by-products, and adjunct reagents (collectively “impurities”) are identified spectroscopically and/or with another physical method, and then associated with a peak position, such as that in a chromatogram, or a spot on a TLC plate. See Strobel, H. A., et al., CHEMICAL INSTRUMENTATION: A SYSTEMATIC APPROACH, 953, 3d ed. (Wiley & Sons, New York 1989). Once a particular impurity has been associated with a peak position, the impurity can be identified in a sample by its relative position in the chromatogram, where the position in the chromatogram is measured in minutes between injection of the sample on the column and elution of the impurity through the detector. The relative position in the chromatogram is known as the “retention time.”

As known by those skilled in the art, the management of process impurities is greatly enhanced by understanding their chemical structures and synthetic pathways and by identifying the parameters that influence the amount of impurities in the final product.

### SUMMARY OF THE INVENTION

In one embodiment, the present invention encompasses isolated caspofungin impurity A (“impurity A”), 1-((4R,5S)-5-(2-Aminoethylamino)-N2-(10,12-dimethyl-1-oxotetradecyl)-4-hydroxy-L-ornithine)-2-L-serine-5-((3R)-3-hydroxy-L-ornithine)-pneumocandin B₀, having the following formula:

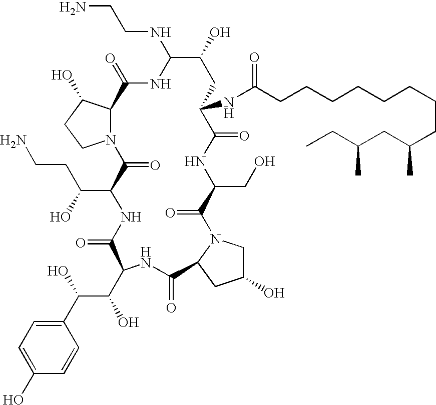

The isolated impurity A of the present invention may be characterized by one or more of: ¹HNMR spectrum having hydrogen chemical shifts at about 0.81, 0.82, 0.83, 1.46, 1.75, 1.84, 3.57, 3.77, 3.90, 3.92, 4.11, 4.17, 4.27, 4.40, 6.66, 6.98 ppm; a ¹³CNMR spectrum having carbon chemical shifts at about 11.00, 19.64, 20.18, 23.84, 25.29, 29.19, 29.50, 31.05, 33.38, 34.65, 35.02, 37.38, 45.60, 49.56, 54.25, 54.55, 55.37, 61.06, 61.68, 62.65, 68.51, 68.70, 69.26, 69.76, 73.13, 73.22, 75.97, 114.69, 128.09, 132.28, 156.59, 166.61, 169.39, 170.63, 170.71, 171.19, 173.68, 174.75 ppm; an MS (ESI⁺) spectrum having peaks at about: m/z=540.319 ([M+2H]²⁺), 1079.630 ([M+H]⁺); a retention time of about 18 min in HPLC analysis, such as the one described herein below; and by a relative retention time of about 0.95.

In one embodiment, the present invention encompasses pure caspofungin having less than about 1.0% by area HPLC of impurity A. Preferably, the pure caspofungin has less than about 0.6%, more preferably less than about 0.05% by area HPLC of impurity A.

In another embodiment, the present invention encompasses a process for purifying caspofungin using a reversed phase chromatography.

In yet another embodiment, the present invention encompasses a process for purifying caspofungin using a preparative HPLC, loaded with a reversed phase resin.

In one embodiment, the present invention further provides the use of impurity A as a reference marker to analyze the purity of caspofungin and salts thereof. The method comprises: a) providing a reference sample comprising caspofungin and salts thereof and impurity A; b) analyzing the reference sample by HPLC and determining the relative retention time of impurity A compared to caspofungin and salts thereof; c) analyzing a sample of caspofungin and salts thereof by HPLC and determining the relative retention times of the contents of the sample as compared to caspofungin and salts thereof; and d) comparing the relative retention times calculated in step c) to the relative retention time calculated in step b) for impurity A, wherein if any of the relative retention times calculated in step c) are substantially the same as the relative retention time of impurity A, impurity A is present in the sample of caspofungin and salts thereof.

In one embodiment, the invention further encompasses a quantification method for determining the amount of impurity A in a caspofungin and salts thereof sample using impurity A as a reference standard. The method comprises: a) measuring by HPLC the area under the peak corresponding to impurity A in a sample of caspofungin and salts thereof having an unknown amount of impurity A; b) measuring by HPLC the area under a peak corresponding to caspofungin and salts thereof in a reference standard having a known amount of impurity A; and c) determining the amount of impurity A in the caspofungin and salts thereof sample by comparing the area calculated in step a) to the area calculated in step b).

In one embodiment, the present invention provides a process for enriching presence of impurity A in a mixture with caspofungin comprising: a) putting the mixture in a column and adding water and acetonitrile to the column; b) collecting samples with enriched impurity A; c) optionally repeating steps a) and b), d) diluting the enriched sample with water to obtain a solution; e) adding the solution to the column; f) adding ethanol containing acetic acid to the column; g) collecting samples with enriched impurity A; h) optionally repeating steps a) and b).

### DETAILED DESCRIPTION OF THE INVENTION

The present invention thus addresses the need in the art for managing impurities in caspofungin and salts thereof, especially caspofungin impurity A, 1-((4R,5S)-5-(2-Aminoethylamino)-N2-(10,12-dimethyl-1-oxotetradecyl)-4-hydroxy-L-ornithine)-2-L-serine-5-((3R)-3-hydroxy-L-ornithine)-pneumocandin B₀ and salts thereof, thus providing caspofungin and salts thereof free of impurities

An HPLC analysis of the commercial dosage form Cancidas® shows that the presence of an impurity referred to as impurity A in the present application, at a level of greater than 1.0% (as demonstrated in example 4).

The present invention is related to the isolated caspofungin impurity A (“impurity A”), 1-((4R,5 S-5-(2-Aminoethylamino)-N2-(10,12-dimethyl-1-oxotetradecyl)-4-hydroxy-L-ornithine)-2-L-serine-5-((3R)-3-hydroxy-L-ornithine)-pneumocandin B₀. It was measured by an HPLC analysis that the crude caspofungin contains less than about 1.0% area by HPLC and even less than 0.6% of impurity A, and after purification using medium pressure reverse phase column chromatograph (RP-MPLC) the impurity levels can be decreased to less than about 0.3% area by HPLC; and even further to below limit of detection using a preparative HPLC method.

Isolation of impurity A by medium pressure reverse phase column chromatograph (RP-MPLC), NMR and mass spectrometric investigations revealed that this impurity is a “serine analogue” of caspofungin.

The present invention also provides the pure form of caspofungin, free of impurity A and the means for preparing such pure caspofungin. The pure caspofungin obtained according to the present invention can be further converted to any pharmaceutically acceptable salt by performing ion-exchange conversion according to known methods in the art. The pure caspofungin obtained according to the present invention is preferably caspofungin diacetate free of caspofungin impurity A diacetate.

As used herein, “isolated”, in reference to caspofungin impurity A, corresponds to impurity A that is physically separated from a reaction mixture. The reaction mixture is typically that which contains caspofungin. For example, the separation can be done by elution from an HPLC column and further drying the impurity A.

In one embodiment, the present invention encompasses isolated caspofungin impurity A (“impurity A”), 1-((4R,5S)-5-(2-Aminoethylamino)-N2-(10,12-dimethyl-1-oxotetradecyl)-4-hydroxy-L-ornithine)-2-L-serine-5-((3R)-3-hydroxy-L-ornithine)-pneumocandin B₀ having the following formula:

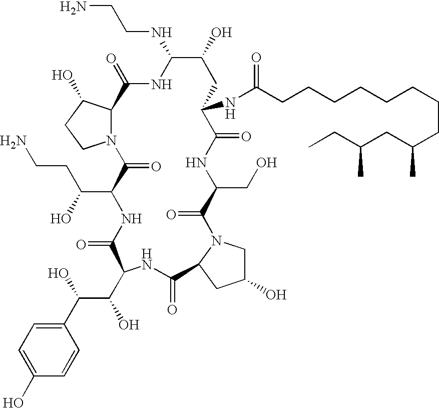

The isolated impurity A of the present invention may be characterized by one or more of: ¹HNMR spectrum having hydrogen chemical shifts at about 0.81, 0.82, 0.83, 1.46, 1.75, 1.84, 3.57, 3.77, 3.90, 3.92, 4.11, 4.17, 4.27, 4.40, 6.66, 6.98 ppm; a ¹³CNMR spectrum having carbon chemical shifts at about 11.00, 19.64, 20.18, 23.84, 25.29, 29.19, 29.50, 31.05, 33.38, 34.65, 35.02, 37.38, 45.60, 49.56, 54.25, 54.55, 55.37, 61.06, 61.68, 62.65, 68.51, 68.70, 69.26, 69.76, 73.13, 73.22, 75.97, 114.69, 128.09, 132.28, 156.59, 166.61, 169.39, 170.63, 170.71, 171.19, 173.68, 174.75 ppm; an MS (ESI⁺) spectrum having peaks at about: m/z=540.319 ([M+2H]²⁺), 1079.630 ([M+H]⁺); a retention time of about 18 min in HPLC analysis, such as the one described herein below, and by a relative retention time of about 0.95.

In one embodiment, the present invention encompasses pure caspofungin having less than about 1.0% by area HPLC of impurity A. Preferably, the pure caspofungin has less than about 0.6%, more preferably less than about 0.05% by area HPLC of impurity A.

In another embodiment, the present invention encompasses a process for purifying caspofungin using a reversed phase chromatography.

Preferably, the caspofungin obtained according to the process described above contains less than about 1.0% by area HPLC of impurity A. Preferably, the pure caspofungin has less than about 0.6%, more preferably less than about 0.3% by area HPLC of impurity A

The reversed phase chromatography used in the process described above can utilize a medium pressure reverse phase column (RP-MPLC) or a high pressure reverse phase column (RP-HPLC). Preferably, the column is RP-MPLC.

The caspofungin is preferably eluted with a mixture of a water immiscible organic solvent and water. The water immiscible organic solvent is preferably acetonitrile or a C₁-C₄ alcohol. More preferably, it is acetonitrile. The volume ratio between the water immiscible solvent and water is preferably about 10:90 to about 40:60 (v/v) of solvent to water. Preferably, the ratio is about 20:80 (v/v) of solvent to water. Preferably, acetic acid is added to the elution mixture.

In yet another embodiment, the present invention encompasses a process for purifying caspofungin using a preparative HPLC, loaded with a reversed phase resin.

Preferably, the caspofungin obtained according to the process described above contains less than about 0.3% by area HPLC of impurity A. Preferably, the pure caspofungin has less than about 0.1%, more preferably, less than about 0.05% by area HPLC of impurity A.

The reversed phase resin used in the process described above is preferably a RP C-18 or RP C-8 resin. More preferably, it is a RP C-18 resin.

The caspofungin is preferably purified with an aqueous buffer and organic buffer. Preferably, the aqueous buffer contains acetic acid and the organic buffer is acetonitrile.

The caspofungin obtained according to the above process is further eluted using lyophilization.

The caspofungin starting material can be obtained according to any method described in the prior art, such as the method described in WO 97/47645, U.S. Pat. No. 5,936,062 or according to example 1 of the present application.

Impurity A is useful as a reference marker for caspofungin and salts thereof. As such, it may be used in order to detect the presence of impurity A in a sample of caspofungin and salts thereof.

In another embodiment, the present invention further provides the use of impurity A as a reference marker to analyze the purity of caspofungin and salts thereof. The method comprises: a) providing a reference sample comprising caspofungin and salts thereof and impurity A; b) analyzing the reference sample by HPLC and determining the relative retention time of impurity A compared to caspofungin and salts thereof; c) analyzing a sample of caspofungin and salts thereof by HPLC and determining the relative retention times of the contents of the sample as compared to caspofungin and salts thereof; and d) comparing the relative retention times calculated in step c) to the relative retention time calculated in step b) for impurity A, wherein if any of the relative retention times calculated in step c) are substantially the same as the relative retention time of impurity A, impurity A is present in the sample of caspofungin and salts thereof.

Impurity A is also useful as a reference standard for caspofungin and salts thereof. As such, it may be used in order to quantify the amount of impurity A in a sample of caspofungin and salts thereof.

In yet another embodiment, the invention further encompasses a quantification method for determining the amount of impurity A in a caspofungin and salts thereof sample using impurity A as a reference standard. The method comprises: a) measuring by HPLC the area under the peak corresponding to impurity A in a sample of caspofungin and salts thereof having an unknown amount of impurity A; b) measuring by HPLC the area under a peak corresponding to caspofungin and salts thereof in a reference standard having a known amount of impurity A; and c) determining the amount of impurity A in the caspofungin and salts thereof sample by comparing the area calculated in step a) to the area calculated in step b).

Preferably, the HPLC methodology used in the above method (for the use of impurity A as reference standard) includes the following steps:
(a) combining a caspofungin and salts thereof sample with a mixture of acetonitrile:water in a ratio of about 1:1, to obtain a solution;
(b) injecting the solution of step (a) into a Synergi Hydro-RP (or similar) column;
(c) eluting the sample from the column at about 20 min using a mixture of buffer, acetonitrile:water (85:15) and methanol:buffer mix (80:18) as an eluent, and
(d) measuring the impurity A content in the relevant sample with a UV detector (preferably at a 225 nm wavelength).

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

NMR Analysis:

NMR spectra were taken on a Bruker DRX 500 instrument (500.13 MHZ ¹H and 125.78 MHZ ¹³C frequency) in DMSO-d₆ solution at 300 K

### Mass Spectrometric Analysis:

Mass spectrum was taken on a Bruker micrOTOFQ mass spectrometer in positive electrospray mode.

### Preparative HPLC Method:

Column: DAISO SP-100-15-ODS-P 100 Å pore size C-18 or DAISO SP-120-15-ODS-ap 120 Å pore size C-18
Eluent: A: 1% acetic acid, 2% acetonitrile in water
B: acetonitrile
gradient program: from 2% acetonitrile to 15% in 10 min. and to 21% acetonitrile in 50 min.
Flow rate: depends on size of the preparative column
Injected volume: depends on the size of the preparative column
Column temperature: room temperature
Detection wavelength: 230 nm
Sample concentration: 1% to 2%
Diluent: aqueous solution as obtained from the reaction

### Impurity Content Determination of Caspofungin by HPLC

### Method 1-

### Column: Synergy Hydro-RP 1 50×4.6 mm, 4 μm

### Eluent:

A: buffer:0.06 M H₃PO₄ pH=2.0/cc NH₃
B: acetonitrile:water 85:15 mixture
C: methanol:buffer 80:18 mixture
   Gradient table:

| t, min | A % | B % | C % |
|---|---|---|---|
| 0 | 52 | 29 | 19 |
| 19 | 31 | 29 | 40 |
| 35 | 12 | 48 | 40 |
| 38 | 52 | 48 | 0 |
| 47 | 12 | 88 | 0 |
| 48 | 52 | 29 | 19 |

Flow rate: 1.0 ml/min
Column temperature: 25° C.
Detection wavelength: 225 nm
Run time: 54 min
Diluent: acetonitrile:water 50:50 mixture
Sample concentration: 2000 μg/ml

### Method 2-

### Column: Hydrosphere C18, 150×4.6 mm, 3 μm

### Eluent:

‘A’: 0.025 M H₃PO₄ pH=2.0/cc.NH₃
‘B’: acetonitrile:(0.025 M H₃PO₄ pH=2.0/cc.NH₃) 70:30
‘C’: methanol:(0.025 M H₃PO₄ pH=2.0/cc.NH₃) 50:50 gradient elution
   Flow rate: 1.0 ml/min
   Injected volume: 10 μl
   Column temperature: 25° C.
   Detection wavelength: 225 nm
   Sample concentration: 2000 μg/ml
   Diluent: (0.025 M NH₄H₂PO₄ pH=6.0/NH₃ buffer):acetonitrile 50:50

### Preparative MPLC Method Used in Isolation of Caspofungin and its Impurity A

300 g reverse phase (SP-100-15-ODS-P; Daiso Co. Ltd.) was charged to a polyethylene coated medium pressure glass column (36×460 mm, BÜCHI). The column was closed and the adsorbent was flooded downstream with mixture of methanol—water (95:5 v/v) with the speed of about 14 ml/min.

Before chromatographic separations described in the examples, the solvent mixture was changed to acetonitrile—water (20:80 v/v).
After chromatographic separations described in the examples, the column was washed with the mixture of methanol—water (95:5 w/v) mixture for refreshment and perfect removal of any material remind on it.
A BÜCHI Pump Module C-610 (P max.: 10 bar) and a BÜCHI Fraction collector B-684 was used for pumping the eluent and fraction collection.

The chemical structure of caspofungin impurity A was determined by ¹³C— and ¹H-NMR spectroscopy and mass spectrometry according to the methods described above. The results are presented in the following table 1 and mass spectrometry FIG. 1:

TABLE 1
¹³C and ¹H chemical shifts

| Chemical shift | | | | |
|---|---|---|---|---|
| δ (ppm) | | Assignment | | |
| ¹³C | ¹H | Residue* | Carbon no. | Multiplicity |
| 11.00 | 0.83 | DMM | C-14 | CH₃ |
| 19.64 | 0.81 | DMM | ^{#}12-CH₃ | CH₃ |
| 20.18 | 0.82 | DMM | ^{#}10-CH₃ | CH₃ |
| 23.84 | 1.75 | — | impurity | CH₃ |
| 25.29 | 1.47 | DMM | C-3 | CH₂ |
| 26.36 | 1.25 | DMM | C-8 | CH₂ |
| 28.54; 28.67; | 1.20-1.30 | DMM | C-4, C-5, C-6, | CH₂ |
| 28.88; 29.06; | | | C-7, C-13 | |
| 29.19 | | | | |
| 29.50 | 1.46 | DMM | C-10 | CH |
| 31.05 | 1.40 | DMM | C-12 | CH |
| 33.38 | 1.84; 2.04 | DiAVA | C-5 | CH₂ |
| 34.65 | 1.80; 1.89 | βOHPro | C-4 | CH₂ |
| 35.02 | 2.13 | TriAVA | C-3 | CH₂ |
| 36.38 | 1.04; 1.27 | DMM | C-9 | CH₂ |
| 37.26 | 1.84; 2.21 | DMM | C-2 | CH₂ |
| 37.38 | 2.69; 2.84 | γOHPro | C-3 | CH₂ |
| 40.09 | 2.72 | TriAVA | (N—CH₂)₂ | CH₂ |
| 44.13 | 0.89; 1.20 | DiAVA | C-4 | CH₂ |
| 44.13 | 2.54; 2.69 | βOHPro | C-5 | CH₂ |
| 45.60 | 3.65; 3.92 | DMM | C-11 | CH₂ |
| 49.56 | 4.11 | TriAVA | C-2 | CH |
| 54.25 | 3.93 | DiOHTyr | C-2 | CH |
| 54.25 | 4.83 | Ser | C-2 | CH |
| 54.55 | 7.74 | DiAVA | C-2 | CH |
| 55.37 | 3.69; 3.80 | γOHPro | C-5 | CH₂ |
| 61.06 | 4.27 | γOHPro | C-2 | CH |
| 61.68 | 3.57 | Ser | C-3 | CH₂ |
| 62.65 | 4.40 | TriAVA | C-5 | CH |
| 68.51 | 4.17 | TriAVA | ^{&}C-4 | CH |
| 68.70 | 3.91 | γOHPro | ^{&}C-4 | CH |
| 69.26 | 4.40 | DiAVA | ^{&}C-3 | CH |
| 69.76 | 3.77 | βOHPro | ^{&}C-2 | CH |
| 73.13 | 3.90 | DiOHTyr | ^{$}C-3 | CH |
| 73.22 | 3.90 | DiOHTyr | ^{$}C-4 | CH |
| 75.97 | 3.90 | βOHPro | C-3 | CH |
| 114.69 | 6.66 | DiOHTyr | C-3′; C-5′ | CH |
| 128.09 | 6.98 | DiOHTyr | C-2′; C-6′ | CH |
| 132.28 | — | DiOHTyr | C-1′ | C |
| 156.59 | — | DiOHTyr | C-4′ | C |
| 166.61 | — | DiAVA | C-1 | C |
| 169.39; | — | DiOHTyr | ^{β}C-1 | C |
| 170.63; | — | Ser | ^{β}C-1 | C |
| 170.71; | — | βOHPro | ^{β}C-1 | C |
| 171.19; | — | γOHPro | ^{β}C-1 | C |
| 173.68; | — | TriAVA | ^{β}C-1 | C |
| 174.75 | — | DMM | ^{β}C-1 | C |

*Abbreviations:
DiOHTyr (3,4-Dihydroxyhomotyrosine),
DiAVA (α,δ-Diamino-β-hydroxyvaleric acid),
βOHPro (3-Hydroxy-proline),
γOHPro (4-Hydroxy-proline),
Ser (Serine),
TriAVA (α,δ-Diamino-δ(β-aminoethyl-amino)-γ-hydroxyvaleric acid),
DMM (10,12-Dimethylmyristate)
^{#,$,&,β}Assignments can be mutually interchanged

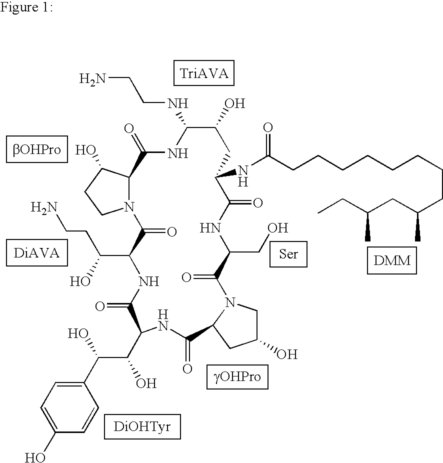

### Example 1

### Preparation of Caspofungin with Controlled Content of Caspofungin Impurity A

One sample of pneumocandin B₀ purified by silica gel column chromatography was transformed to caspofungin according to the following examples:

### Example 1A

### Preparation of 4-methoxyphenylthio-pneumocandin B₀

Pneumocandin B₀ (25.2 g) (assay: 89.3%; HPLC purity: 91.0 A %) was suspended in acetonitrile (630 ml) in a jacketed reactor fitted with thermometer, nitrogen inlet and mechanical stirrer.

The mixture was cooled to −15° C. by means of a thermostat, and 4-methoxythiophenol (5.88 g) was added in one portion. Trifluoroacetic acid (117.9 g) was added dropwise in about 20 min keeping the temperature between −10÷−15° C. The mixture was stirred at −15° C. for 22 h and quenched by addition of water (1260 ml) at a temperature below 0° C. in about 60 min. The mixture was stirred at about 0° C. for 1 h then the precipitated solid was collected, washed twice with acetonitrile—water (1:3 v/v) (140 and 140 ml) and twice with acetonitrile (105 and 70 ml) to afford the product 23.97 g (85.2%) after drying in vacuum at less than 40° C. for 24 h in the HPLC purity of 78.8 A % and assay of 72.2%.

### Example 1B

### Preparation of 4-methoxyphenylthio-pneumocandin B₀ amine

4-Methoxyphenylthio-pneumocandin B₀ (14.0 g) was suspended in tetrahydrofuran (500 ml) then phenylboronic acid (2.31 g) was added, and the mixture was stirred at less than 40° C. until obtaining a solution (4 h).

Molecular sieve of 3 Å (50 g) was then added to the mixture and was allowed to stand at room temperature for about 16 h to decrease the water content (LT 150 ppm).

The molecular sieve was removed, washed with THF (50 ml) and the filtrate was charged to a jacketed reactor fitted with nitrogen inlet, thermometer and a thermostat. The solution was cooled to −5° C. and borane-dimethylsulfide complex (3.86 g/90% pure/) was added in about 15 min at 0÷−5° C. resulting in a dense gelatinous mixture in 30 min after addition which was stirred at about −5° C. for 10 h.

The reaction mixture was cooled to −15° C., and quenched by addition of 2N aqueous hydrochloric acid solution (8 ml) at (−10)-(−15)° C. in about 15 min resulting in a clear solution.

The quenched mixture was stored in a freezer at about −15° C. overnight, then was diluted with water (2200 ml).

The diluted solution was filtered through a sintered glass filter and charged onto a 295 g reverse phase (LiChroprep RP-18, Merck) medium pressure column (36×460 mm) with the speed of about 18 ml/min. The column was washed with acetonitrile—water (20:80 v/v; 1800 ml; 18 ml/min) and the product was eluted with acetonitrile—water (40:60 v/v; about 14 ml/min). Fractions of 200 ml each were collected by means of a fraction collector and analyzed by TLC, than the fractions showing the presence of the product, by HPLC.

The rich cuts (>88 A %) were combined, diluted with water and charged to a 125 g of a reverse phase column (LiChroprep RP-18, Merck).

The product was eluted with methanol by means of gravitation, collecting 5×120 ml fraction which were analyzed by HPLC. The suitable fractions were combined and concentrated on a rotary evaporator at a temperature of less than 30° C. and the product was precipitated by addition of acetonitrile.

The mixture was cooled to 2-8° C., the solid was collected, washed with acetonitrile (20 ml) and dried in a vacuum oven at room temperature for 24 h to yield 4.82 g (35.2%) of the product in a HPLC purity of 96.8 A % and assay of 91.9%.

### Example 1 C

### Preparation of Caspofungin Diacetate

4-Methoxyphenylthio-pneumocandin B₀ amine (4.34 g) was added to ethylenediamine (18.5 ml) under nitrogen while stirring and cooling at 15-25° C.

The mixture was stirred at room temperature for 6 h then it was diluted with methanol (24 ml) while cooling with ice-water at 15-25° C. The mixture of water (90 ml) and acetic acid (24 ml) was added under the same condition, and finally, the pH of the mixture was adjusted to 6-7 by addition of acetic acid (8 ml).

The neutralized mixture was diluted with water (310 ml), washed with toluene (3×47 ml) and filtered through a G-4 sintered glass filter.

The solution contained 0.30% of caspofungin Impurity A on the basis of HPLC analysis.

The solution was charged to a 300 g reverse phase (SP-100-15-ODS-P; Daiso Co. Ltd.) medium pressure column (36×460 mm) with the speed of about 14 ml/min, and the product was eluted with acetonitrile—water (20:80 v/v+0.01% acetic acid; 14 ml/min). Fractions of 100 ml each were collected and analyzed by TLC, then the fractions showing the presence of caspofungin, by HPLC

The first fraction containing 2.54% of Impurity A was put aside for isolation of the impurity, the remaining rich cuts (>99.0 A % for caspofungin (HPLC) were combined and lyophilized to afford 3.19 g (71.7%) caspofungin acetate as a cotton-like white solid.

The Caspofungin Impurity A content of the product was 0.16% on the basis of HPLC analysis.

### Example 2

### Preparation of Caspofungin Diacetate Free of Caspofungin Impurity A

The crude product (caspofungin solution) was produced as described in Example 1C via reaction between 4-Methoxyphenylthio-pneumocandin B₀ amine and ethylenediamine. The reaction was carried out under nitrogen at room temperature for 6 h then the reaction mixture was diluted with methanol while cooling with ice-water at 15-25° C. The mixture of water and acetic acid was added under the same condition, and finally, the reaction solution was diluted with water and neutralized to pH about 4 to 5 by addition of acetic acid.

The solution was loaded to the preparative HPLC (loaded with RP C-18 resin or similar) and was purified using aqueous buffer containing acetic acid and acetonitrile.

The purified fractions (>99.0% pure; each impurity <0.1% including impurity A) were collected and loaded to the lyophilizer to obtain final dry powder of Caspofungin diacetate containing <0.05% impurity A as determined by HPLC analysis.

### Example 3

### Enrichment of Caspofungin Impurity A

Fractions of several MPLC purifications described in example 1 C enriched in caspofungin impurity A were collected and diluted with the same amount of water. The diluted solution was charged to the same column described in example 1 C. The column was washed with the mixture of acetonitrile and water (20:80, v/v; about 14 ml/min). Fractions of 100 ml each were collected by means of a fraction collector and analyzed by HPLC.

After obtaining fractions rich in impurity A (17-19), 0.1% of acetic acid was added to the eluent and continue elution to obtain fractions rich in caspofungin. Selecting the best fractions, and reprocessing the less pure ones, 3 additional chromatographic circle performed similar way described above. The rich fractions containing impurity A (>73%) were collected and diluted with the same amount of water.

The diluted solution was charged onto the same column described above, and the product was eluted with ethanol containing 0.025% acetic acid (14 ml/min; 56 ml fractions each).

Fractions (5-9) containing the impurity A were collected, concentrated to about 20 ml diluted with 50 ml of water and charged to a charged to a 23 g reverse phase (SP-100-15-ODS-P; Daiso Co. Ltd.) medium pressure column (12×230 mm) with the speed of about 1 ml/min, and the product was eluted with acetonitrile—water (20:80 v/v+0.1% acetic acid; 2 ml/min).

Fractions of 100 ml each were collected and analyzed by HPLC. The rich fractions containing the product (>90%) were collected and lyophilized to afford 120 mg of caspofungin impurity A in the purity of 94.0 A % (HPLC).

### Example 4

### Analysis of Cancidas® Lyophilized powder

Cancidas® tablets were analyzed according to the following HPLC method, and found to contain 1.11-1.26% area by HPLC, of impurity A.

### Column: Synergy Hydro-RP 150×4.6 mm, 4 μm

### Eluent:

A: buffer:0.06 M H₃PO₄ pH=2.0/cc NH₃
B: acetonitrile:water 85:15 mixture
C: methanol:buffer 80:18 mixture
   Gradient table:

| t, min | A % | B % | C % |
|---|---|---|---|
| 0 | 52 | 29 | 19 |
| 19 | 31 | 29 | 40 |
| 35 | 12 | 48 | 40 |
| 38 | 52 | 48 | 0 |
| 47 | 12 | 88 | 0 |
| 48 | 52 | 29 | 19 |

Flow rate: 1.0 ml/min
Column temperature: 25° C.
Detection wavelength: 225 nm
Run time: 54 min
Diluent: acetonitrile:water 50:50 mixture
Sample concentration: 2000 μg/ml

1. Isolated caspofungin impurity A, 1-((4R,5S)-5-(2-Aminoethylamino)-N2-(10,12-dimethyl-1-oxotetradecyl)-4-hydroxy-L-ornithine)-2-L-serine-5-((3R)-3-hydroxy-L-ornithine)-pneumocandin Bo, having the following formula:

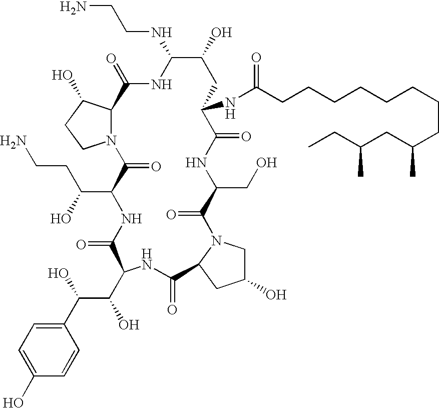

2. The isolated caspofungin impurity of claim 1 characterized by data selected from: ¹HNMR spectrum having hydrogen chemical shifts at about 0.81, 0.82, 0.83, 1.46, 1.75, 1.84, 3.57, 3.77, 3.90, 3.92, 4.11, 4.17, 4.27, 4.40, 6.66, 6.98 ppm; a ¹³CNMR spectrum having carbon chemical shifts at about 11.00, 19.64, 20.18, 23.84, 25.29, 29.19, 29.50, 31.05, 33.38, 34.65, 35.02, 37.38, 45.60, 49.56, 54.25, 54.55, 55.37, 61.06, 61.68, 62.65, 68.51, 68.70, 69.26, 69.76, 73.13, 73.22, 75.97, 114.69, 128.09, 132.28, 156.59, 166.61, 169.39, 170.63, 170.71, 171.19, 173.68, 174.75 ppm; an MS (ESI⁺) spectrum having peaks at about: m/z=540.319 ([M+2H]²⁺), 1079.630 ([M+H]⁺) and by a retention time of about 18 min in HPLC analysis, and by a relative retention time of about 0.95.

3. Pure caspofungin having less than about 1.0% by area HPLC of impurity A.

4. The pure caspofungin of claim 3 having less than about 0.6% by area HPLC of impurity A.

5. The pure caspofungin of claim 4 having less than about 0.05% by area HPLC of impurity A.

6. A process of purifying Caspofungin comprising performing chromatography with a reversed phase chromatography.

7. The process of claim 6, wherein the reversed phase chromatography is medium pressure reverse phase column (RP-MPLC) or a high pressure reverse phase column (RP-HPLC).

8. The process of claim 6, wherein the reversed phase chromatography is medium pressure reverse phase column (RP-MPLC).

9. The process of claim 6, wherein the caspofungin is eluted with a mixture of a water immiscible organic solvent and water.

10. The process of claim 9, wherein the water immiscible organic solvent is selected from the group consisting of: acetonitrile and a C₁-C₄ alcohol.

11. The process of claim 9, wherein the water immiscible organic solvent is acetonitrile.

12. The process of claim 9, wherein the volume ratio between the water immiscible solvent and water is preferably about 10:90 to about 40:60 (v/v) of solvent to water.

13. The process of claim 9, wherein the volume ratio between the water immiscible solvent and water is preferably about 20:80 (v/v) of solvent to water.

14. The process of claim 9, wherein acetic acid is added to the elution mixture.

15. The process of claim 14, wherein 0.025% to about 1.0% are added to the

16. The process of claim 6 wherein the obtained Caspofungin contains less than 1.0% by area HPLC of impurity A.

17. The process of claim 6 wherein the obtained Caspofungin contains less than 0.6% by area HPLC of impurity A.

18. The process of claim 6 wherein the obtained Caspofungin contains less than 0.3% by area HPLC of impurity A.

19. A process for purifying caspofungin comprising performing preparative HPLC loaded with a reversed phase resin.

20. The process of claim 19, wherein a reverse resin C-18 or C-8 is used.

21. The process of claim 20, wherein a reverse phase resin is C-18

22. The process of claim 19, wherein the obtained caspofungin contains less than about 0.3% by area HPLC of impurity A.

23. The process of claim 22, wherein the obtained caspofungin contains less than about 0.1% by area HPLC of impurity A.

24. The process of claim 22, wherein the obtained caspofungin contains less than about 0.05% by area HPLC of impurity A.

25. The process of claim 19, wherein the caspofungin is purified with an aqueous buffer and organic buffer.

26. The process of claim 25, wherein the aqueous buffer contains acetic acid and the organic buffer is acetonitrile.

27. A process for enriching presence of impurity A in a mixture with caspofungin comprising:
a) putting the mixture in a column and adding water and acetonitrile to the column;
b) collecting samples with enriched impurity A;
c) optionally repeating steps a) and b),
d) diluting the enriched sample with water to obtain a solution;
e) adding the solution to the column;
f) adding ethanol containing acetic acid to the column;
g) collecting samples with enriched impurity A;
h) optionally repeating steps a) and b).

28. A method of using caspofungin impurity A as a reference marker to analyze the purity of caspofungin comprising:
a) providing a reference sample comprising caspofungin and caspofungin impurity A;
b) analyzing the reference sample by HPLC and determining the relative retention time caspofungin impurity A compared to caspofungin;
c) analyzing a sample of caspofungin by HPLC and determining the relative retention times of the contents of the sample as compared to caspofungin; and
d) comparing the relative retention times calculated in step c) to the relative retention time calculated in step b) for caspofungin impurity A, wherein if any of the relative retention times calculated in step c) are substantially the same as the relative retention time of caspofungin impurity A then caspofungin impurity A is present in the sample of caspofungin.

29. A method of using caspofungin impurity A as a reference standard for determining the amount of caspofungin impurity A in a caspofungin sample comprising:
a) measuring by HPLC the area under the peak corresponding to caspofungin impurity A in a sample of caspofungin having an unknown amount of caspofungin impurity A;
b) measuring by HPLC the area under a peak corresponding to caspofungin impurity A in a reference standard comprising a known amount of caspofungin impurity A; and
c) determining the amount of caspofungin impurity A in the caspofungin sample by comparing the area calculated in step a) to the area calculated in step b).

30. A quantification method for determining the amount of caspofungin impurity A in a caspofungin sample comprising:
a) measuring by HPLC the area under the peak corresponding to caspofungin impurity A in a sample of caspofungin having an unknown amount of caspofungin impurity A;
b) measuring by HPLC the area under a peak corresponding to caspofungin in a reference standard having a known amount of caspofungin; and
c) determining the amount of caspofungin impurity A in the caspofungin sample by comparing the area calculated in step a) to the area calculated in step b).

31. The method of claim 30, wherein the HPLC method used includes the following steps:
(e) combining a caspofungin and salts thereof sample with a mixture of acetonitrile:water in a ratio of about 1:1, to obtain a solution;
(f) injecting the solution of step (a) into a Synergi Hydro-RP (or similar) column;
(g) eluting the sample from the column at about 20 min using a mixture of buffer, acetonitrile:water (85:15) and methanol:buffer mix (80:18) as an eluent, and
(h) measuring the impurity A content in the relevant sample with a UV detector (preferably at a 225 nm wavelength).

32. A pharmaceutical composition comprising caspofungin, or any pharmaceutical acceptable salts thereof, of claim 3, and at least one pharmaceutically acceptable excipient.

33. The pharmaceutical composition of claim 32, wherein the pharmaceutical composition is prepared by a process comprising combining the caspofungin or any pharmaceutical acceptable salts thereof with at least one pharmaceutically acceptable excipient.

34. A method of treatment comprising administering to a mammal in need thereof a pharmaceutical composition comprising caspofungin, or any pharmaceutical acceptable salts thereof of claim 3, for the treatment of systemic fungal infections caused by Candida, Aspergillus, Histoplasma, Coccidioides and Blastomyces, and for the treatment and prevention of infections caused by Pneumocystis carinii.
